(19) United States
(12) Patent Application Publication
(10) Pub. No.: US 2026/0054081 A1
(43) Pub. Date: Feb. 26, 2026

(54) Reducing Electrosensation Whilst Treating a Subject Using Alternating Electric Fields by Pairing Transducer Arrays Together

(71) Applicant: Novocure GmbH, Baar (CH)

(72) Inventor: Moshe GILADI, Haifa (IL)

(73) Assignee: Novocure GmbH, Baar (CH)

(21) Appl. No.: 19/372,670

(22) Filed: Oct. 29, 2025

Related U.S. Application Data
(62) Division of application No. 18/128,644, filed on Mar. 30, 2023, now Pat. No. 12,478,793.
(60) Provisional application No. 63/325,438, filed on Mar. 30, 2022.

(51) Int. Cl. A61N 1/40; A61N 1/04; A61N 1/30; A61N 1/36

(52) U.S. Cl. CPC A61N 1/40; A61N 1/0476; A61N 1/30; A61N 1/36002; A61N 1/36034

(57) ABSTRACT

When treating a subject using alternating electric fields (e.g., using TTFields to treat a tumor, or using alternating electric fields to increase the permeability of the blood brain barrier), some subjects experience an unpleasant electrosensation effect. This electrosensation can be reduced or eliminated by increasing the area of the transducer arrays that is active during certain times in the treatment. In some embodiments, this is accomplished by applying an AC signal between two pairs of transducer arrays at certain times (as opposed to the prior art approach of applying an AC signal between two individual transducer arrays).

### CROSS REFERENCE TO RELATED APPLICATIONS

This application is a divisional of U.S. application Ser. No. 18/128,644, filed Mar. 30, 2023, which claims the benefit of U.S. Provisional Application 63/325,438, filed Mar. 30, 2022, each of which is incorporated herein by reference in its entirety.

### BACKGROUND

Tumor Treating Fields, or TTFields, are alternating electric fields within the intermediate frequency range (e.g., 100-500 kHz) that inhibit cancer cell growth. This non-invasive treatment targets solid tumors and is described in U.S. Pat. No. 7,565,205, which is incorporated herein by reference in its entirety. 200 kHz TTFields are FDA approved for the treatment of glioblastoma (GBM), and may be delivered, for example, via the prior art Optune® system, which is depicted schematically in FIG. 1. The TTFields are delivered to patients via four transducer arrays 10 (i.e., electrode arrays) that are placed on the patient's skin in close proximity to a tumor. Each transducer array 10 includes a plurality (e.g., between 9 and 20) capacitively coupled electrode elements, each of which has an electrically conductive substrate with a dielectric layer disposed thereon.

Alternating electric fields at frequencies between 50 kHz and 1 MHz can also be used to treat medical conditions other than tumors. For example, as described in U.S. Pat. No. 10,967,167 (which is incorporated herein by reference in its entirety), alternating electric fields e.g., at 50-200 kHz can increase the permeability of the blood brain barrier (BBB) so that, e.g., chemotherapy drugs can reach the brain. And as described in U.S. Pat. No. 11,103,698 (which is incorporated herein by reference in its entirety), alternating electric fields e.g., at 50-500 kHz can increase the permeability of cell membranes so that large molecules can traverse cell membranes.

When Optune® is used to treat glioblastoma, one pair of arrays (10L, 10R) is positioned to the left and right of the tumor, and the other pair of arrays (10A, 10P) is positioned anterior and posterior to the tumor. The Optune® signal generator 95 (a) applies an AC voltage between arrays 10L, 10R for 1 second, which induces an electric field in one direction through the tumor; then (b) applies an AC voltage between the arrays 10A, 10P for 1 second, which induces an electric field in another direction through the tumor; then repeats that two-step sequence (a) and (b) for the duration of the treatment.

### SUMMARY OF THE INVENTION

One aspect of this application is directed to a first method for treating a tumor in a target region and delivering a substance across a biological barrier in the target. The first method comprises applying an alternating voltage at a first frequency between a first set of one or more electrode elements positioned at a first side of the target region and a second set of one or more electrode elements positioned at a second side of the target region, wherein the first side and the second side are on opposite sides of the target region. The first method also comprises applying an alternating voltage at a second frequency between a third set of one or more electrode elements positioned at a third side of the target region and a fourth set of one or more electrode elements positioned at a fourth side of the target region, wherein the third side and the fourth sides are on opposite sides of the target region, wherein the third side is disposed circumferentially between the first side and the second side, and wherein the fourth side is disposed circumferentially between the first side and the second side. And the first method also comprises applying an alternating voltage at a third frequency between (i) both the first and third sets of one or more electrode elements and (ii) both the second and fourth sets of one or more electrode elements, wherein the third frequency is lower than the first frequency and the third frequency is lower than the second frequency. The alternating voltage at the first frequency, the alternating voltage at the second frequency, and the alternating voltage at the third frequency are each applied during different intervals of time.

Some instances of the first method further comprise positioning the first set of one or more electrode elements at the first side of the target region; positioning the second set of one or more electrode elements at the second side of the target region; positioning the third set of one or more electrode elements at the third side of the target region; and positioning the fourth set of one or more electrode elements at the fourth side of the target region.

In some instances of the first method, the first frequency is selected so that an electric field that is induced in the target region due to application of the alternating voltage at the first frequency between the first set of one or more electrode elements and the second set of one or more electrode elements will have an anti-mitotic effect. The second frequency is selected so that an electric field that is induced in the target region due to application of the alternating voltage at the second frequency between the third set of one or more electrode elements and the fourth set of one or more electrode elements will have an anti-mitotic effect. And the third frequency is selected so that an electric field that is induced in the target region due to application of the alternating voltage at the third frequency between (a) both the first and third sets of one or more electrode elements and (b) both the second and fourth sets of one or more electrode elements will increase permeability of the biological barrier in the target region. Optionally, in these instances, the first frequency and the second frequency may be the same.

In some instances of the first method, the first and second frequencies are each between 50 kHz and 1 MHz, and the third frequency is between 50 kHz and 300 kHz. In some instances of the first method, the alternating voltage at the third frequency is applied for at least 24 hours.

In some instances of the first method, the steps of (a) applying the alternating voltage at the first frequency between the first set of one or more electrode elements and the second set of one or more electrode elements and (b) applying the alternating voltage at the second frequency between the third set of one or more electrode elements and the fourth set of one or more electrode elements are repeated in an alternating sequence at least 10,000 times.

Another aspect of this application is directed to a first apparatus for treating a tumor in a target region and promoting delivery of a substance across a biological barrier in the target region. The first apparatus comprises at least one signal generator, a bank of switches, and a controller. The at least one signal generator is configured to generate an alternating voltage at a first frequency and to generate an alternating voltage at a second frequency, wherein the second frequency is lower than the first frequency. The bank of switches has at least one control input, a first output terminal, a second output terminal, a third output terminal, and a fourth output terminal. The bank of switches inputs the alternating voltage at the first frequency, and inputs the alternating voltage at the second frequency. The bank of switches is configured to, in response to a first state of the at least one control input, route the alternating voltage at the first frequency so that the alternating voltage at the first frequency appears between the first output terminal and the second output terminal. The bank of switches is also configured to, in response to a second state of the at least one control input, route the alternating voltage at the first frequency so that the alternating voltage at the first frequency appears between the third output terminal and the fourth output terminal. And the bank of switches is also configured to, in response to a third state of the at least one control input, route the alternating voltage at the second frequency so that the alternating voltage at the second frequency appears between (a) both the first output terminal and the third output terminal and (b) both the second output terminal and the fourth output terminal. The controller is programmed to, at different intervals of times, (i) set the at least one control input to the first state, (ii) set the at least one control input to the second state, and (iii) set the at least one control input to the third state.

In some embodiments of the first apparatus, the first frequency is selected to provide an anti-mitotic effect, and the second frequency is selected to increase a permeability of a biological barrier. In some embodiments of the first apparatus, the first frequency is between 50 kHz and 1 MHz, and the second frequency is between 50 kHz and 300 kHz. In some embodiments of the first apparatus, the controller is programmed to (i) set the at least one control input to the first state and (ii) set the at least one control input to the second state in an alternating sequence at least 10,000 times. In some embodiments of the first apparatus, the controller is programmed to, after repeating (i) and (ii) at least 10,000 times, set the at least one control input to the third state and subsequently leave the at least one control input in the third state for at least 12 hours.

Another aspect of this application is directed to a second method for applying an alternating electric field to a target region in a subject's body using a first set of one or more electrode elements positioned at a first side of the target region, a second set of one or more electrode elements positioned at a second side of the target region, a third set of one or more electrode elements positioned at a third side of the target region, and a fourth set of one or more electrode elements positioned at a fourth side of the target region. The second method comprises (a) applying an alternating voltage at a first frequency between (i) both the first and third sets of one or more electrode elements and (ii) both the second and fourth sets of one or more electrode elements; and (b) applying an alternating voltage at a second frequency between (i) both the first and fourth sets of one or more electrode elements and (ii) both the second and third sets of one or more electrode elements. The first side and the second side are on opposite sides of the target region. The third side and the fourth side are on opposite sides of the target region. The third side is disposed circumferentially between the first side and the second side. And the fourth side is disposed circumferentially between the first side and the second side. Steps (a) and (b) are repeated in an alternating sequence at least ten times.

Some instances of the second method further comprise positioning the first set of one or more electrode elements on or in the subject's body; positioning the second set of one or more electrode elements on or in the subject's body; positioning the third set of one or more electrode elements on or in the subject's body; and positioning the fourth set of one or more electrode elements on or in the subject's body.

In some instances of the second method, the first frequency and the second frequency are each between 50 kHz and 1 MHz. In some instances of the second method, the first frequency and the second frequency are the same.

Another aspect of this application is directed to a second apparatus for applying signals to a set of electrodes. The second apparatus comprises at least one signal generator, a bank of switches, and a controller. The at least one signal generator is configured to generate an alternating voltage. The bank of switches has at least one control input, a first output terminal, a second output terminal, a third output terminal, and a fourth output terminal; and the bank of switches inputs the alternating voltage at the first frequency. The bank of switches is configured to, in response to a first state of the at least one control input, route the alternating voltage so that the alternating voltage appears between (a) both the first output terminal and the third output terminal and (b) both the second output terminal and the fourth output terminal. The bank of switches is also configured to, in response to a second state of the at least one control input, route the alternating voltage so that the alternating voltage appears between (a) both the first output terminal and the fourth output terminal and (b) both the second output terminal and the third output terminal. And the controller is programmed to repeat the following steps in an alternating sequence at least ten times (i) set the at least one control input to the first state, and (ii) set the at least one control input to the second state.

In some embodiments of the second apparatus, the alternating voltage has a frequency between 50 kHz and 1 MHz.

Another aspect of this application is directed to a third method of applying an alternating electric field to a body part of a subject, the body part having a longitudinal axis. The third method comprises applying an alternating voltage between a first set of one or more electrode elements positioned on or in a first side of the body part and a second set of one or more electrode elements positioned on or in a second side of the body part. The first side and the second side are on opposite sides of the longitudinal axis. The first set of one or more electrode elements subtends a first azimuthal angle of at least 120° with respect to the longitudinal axis, within which the coverage by electrode elements is at least one third. And the second set of one or more electrode elements subtends a second azimuthal angle of at least 120° with respect to the longitudinal axis, within which the coverage by electrode elements is at least one third.

In some instances of the third method, the first set of one or more electrode elements is a set of capacitively coupled electrode elements, and the second set of one or more electrode elements is a set of capacitively coupled electrode elements.

In some instances of the third method, the alternating voltage has a frequency of less than 1 MHz. In some instances of the third method, the alternating voltage has a frequency of less than 190 kHz. In some instances of the third method, the alternating voltage is applied for at least 24 hours. In some instances of the third method, the first azimuthal angle is at least 135°, and the second azimuthal angle is at least 135°.

In some instances of the third method, the coverage by the electrode elements within the first azimuthal angle is at least one half, and the coverage by the electrode elements within the second azimuthal angle is at least one half.

In some instances of the third method, the body part is a head, a collective area of the first set of one or more electrode elements is at least 40 cm², and a collective area of the second set of one or more electrode elements is at least 40 cm².

Another aspect of this application is directed to a fourth method of applying an alternating electric field to a subject's head, the head having a longitudinal axis and a circumference. The fourth method comprises applying an alternating voltage between a first set of one or more electrode elements positioned on or in a first side of the subject's head and a second set of one or more electrode elements positioned on or in a second side of the subject's head. The first side and the second side are on opposite sides of the longitudinal axis. The first set of one or more electrode elements extends for a first length of at least 15 cm along the circumference, within which the coverage by electrode elements is at least one third. And the second set of one or more electrode elements extends for a second length of at least 15 cm along the circumference, within which the coverage by electrode elements is at least one third.

In some instances of the fourth method, the first set of one or more electrode elements is a set of capacitively coupled electrode elements, and the second set of one or more electrode elements is a set of capacitively coupled electrode elements.

In some instances of the fourth method, the alternating voltage has a frequency of less than 1 MHz. In some instances of the fourth method, the alternating voltage has a frequency of less than 190 kHz. In some instances of the fourth method, the alternating voltage is applied for at least 24 hours.

In some instances of the fourth method, the coverage by the electrode elements within the first length is at least one half, and the coverage by the electrode elements within the second length is at least one half.

In some instances of the fourth method, a collective area of the first set of one or more electrode elements is at least 40 cm², and a collective area of the second set of one or more electrode elements is at least 40 cm².

### BRIEF DESCRIPTION OF THE DRAWINGS

FIG. 1 is a schematic representation of the prior art Optune® system.

FIGS. 2A-2D depict a set of transducer arrays that are used to apply alternating electric field treatment to a subject's head.

FIG. 3A is a schematic representation of the electric field that is induced when an AC voltage is applied between the left and right transducer arrays.

FIG. 3B is a schematic representation of the electric field that is induced when an AC voltage is applied between the anterior and posterior transducer arrays.

FIG. 4 is a block diagram of an embodiment that applies one phase of an AC signal to two of the transducer arrays simultaneously, and applies the other phase of the AC signal to the other two transducer arrays simultaneously.

FIG. 5A is a schematic representation of the electric field that is induced when an AC voltage is applied between two transducer arrays (anterior+left) and the remaining two transducer arrays.

FIG. 5B is a schematic representation of the electric field that is induced when an AC voltage is applied between two transducer arrays (anterior+right) and the remaining two transducer arrays.

FIG. 6 is a timing diagram of commands for inducing electric fields in the FIG. 5A direction and the FIG. 5B direction at different times.

FIG. 7 is a block diagram of another embodiment that applies one phase of an AC signal to two of the transducer arrays simultaneously, and applies the other phase of the AC signal to the other two transducer arrays simultaneously.

FIG. 8A is a timing diagram of commands for inducing electric fields in the FIG. 3A direction, the FIG. 3B direction, and the FIG. 5A direction at different times.

FIG. 8B is a timing diagram of commands for inducing electric fields in the FIG. 3A direction, the FIG. 3B direction, and the FIG. 5B direction at different times.

Various embodiments are described in detail below with reference to the accompanying drawings, wherein like reference numerals represent like elements.

### DESCRIPTION OF THE PREFERRED EMBODIMENTS

When treating a subject using alternating electric fields, higher amplitudes are strongly associated with higher efficacy of treatment. However, as the amplitude of the alternating electric field increases, and/or as the frequency of the alternating electric field decreases (e.g., to the vicinity of 100 kHz), some subjects experience an electrosensation effect when the alternating electric field switches direction. This electrosensation could be, for example, a vibratory sensation, paresthesia, and/or a twitching or contraction sensation of muscle fibers. The electrosensation is believed to originate from interactions between the alternating electric fields and nerve cells (i.e., neurons) that are positioned near or adjacent to the transducer arrays. And these sensations may discourage some subjects from continuing their treatment using alternating electric fields.

This application describes a variety of approaches for reducing or eliminating electrosensation while a subject is being treated with alternating electric fields. These approaches rely on increasing the area of the transducer arrays that is active during certain times in the treatment. In some embodiments, this is accomplished by applying an AC signal between two pairs of transducer arrays at certain times (as opposed to the prior art approach of applying an AC signal between two individual transducer arrays).

Assume that an AC current at a given frequency is being coupled through an electrode into a subject's body through a pair of electrodes that are positioned on the subject's skin. When the current density (i.e., the current through any given electrode divided by the area of that electrode) is below a threshold value (which may vary from person to person), electrosensation does not occur. But when the current density rises above that threshold, electrosensation starts to occur, and will become more noticeable when the current density is increased further.

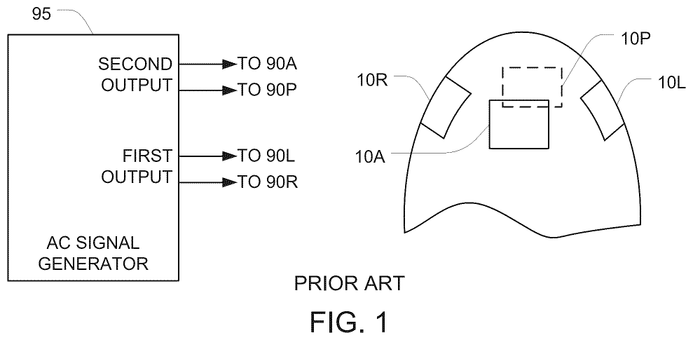

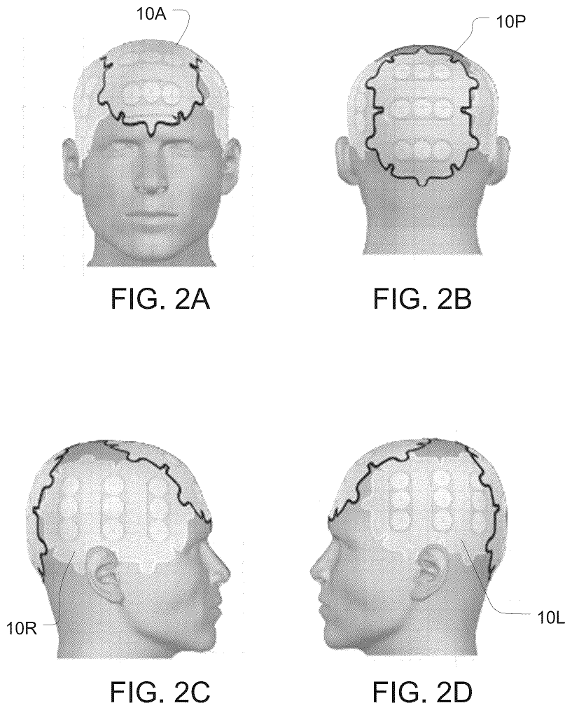

An example that uses numeric values will be helpful to illustrate this point. Assume that the prior art Optune® system depicted in FIG. 1 is being used to treat a glioblastoma in a subject's head using TTFields at 200 kHz. FIGS. 2A-2D depict the Optune® transducer arrays that are used to apply the treatment to the subject's head. Each of these transducer arrays includes nine round electrode elements that have a diameter of 2 cm (i.e., a radius of 1 cm). The total area of all the electrode elements within any given transducer array is therefore 9×3.14×12=28 cm².

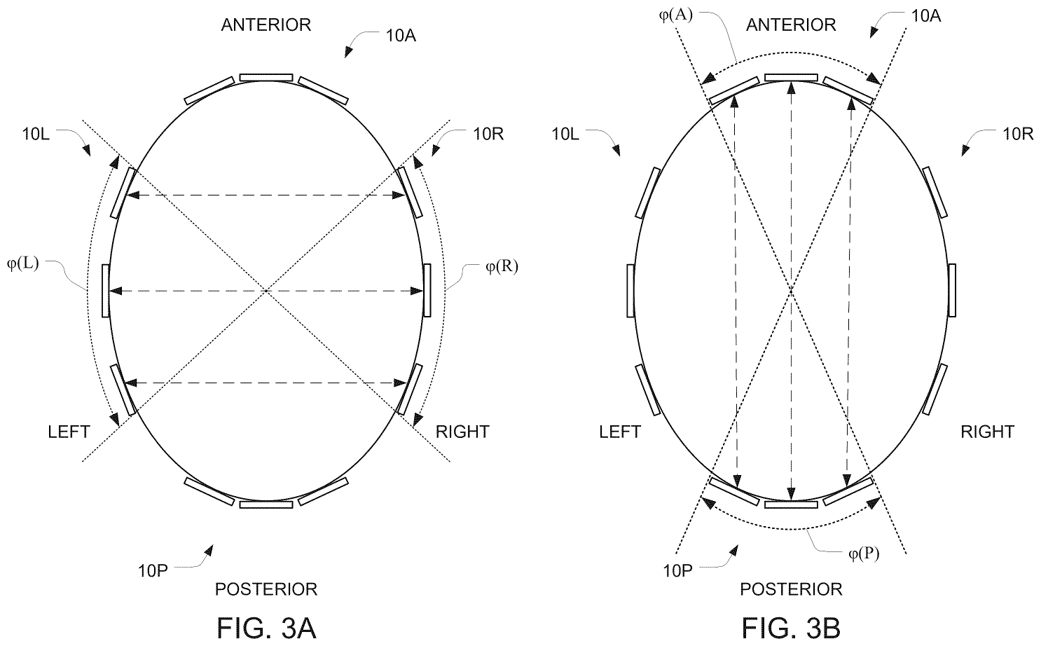

Let us further assume that the output current of the prior art Optune system is 2 A. As explained above, during some intervals of time an AC voltage is applied only between arrays 10L and 10R, which induces an electric field in the subject's head with field lines that run, on average, from left to right (i.e., the direction of the dashed lines in FIG. 3A); and during other intervals of time an AC voltage is applied only between arrays 10A and 10P, which induces an electric field in the subject's head with field lines that run, on average, from front to back (i.e., the direction of the dashed lines in FIG. 3B). During the former intervals, the current density at each of the transducer arrays 10L and 10R will be 2 A÷28 cm²=71 mA/cm². And during the latter intervals, the current density at each of the transducer arrays 10L and 10R will be 2 A÷28 cm²=71 mA/cm². (Note that in reality, the electric field lines will not be straight. But straight dashed lines are nevertheless used in FIGS. 3A/B and 5A/B to represent the general direction of the field lines.)

Now let us assume that the threshold at which electrosensation starts to occur for a particular subject (referred to hereinafter as “subject #1”) at a particular frequency is 60 mA/cm². Because the current density at each of the transducer arrays in this example is 71 mA/cm² (i.e., higher than the threshold for subject #1), subject #1 will experience electrosensation. One approach for ameliorating this situation would be to reduce the current e.g., to 1.5 A, which would reduce the current density to 1.5 A÷28 cm²=54 mA/cm², which is below the electrosensation threshold for subject #1. But this approach is sub-optimal because lower currents typically reduce the efficacy of the treatment.

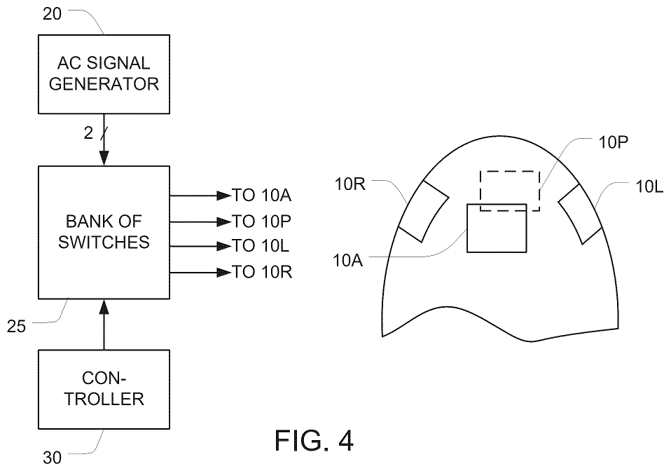

FIG. 4 is a block diagram of an embodiment that uses a different approach for reducing the current density at each of the transducer arrays that does not reduce the overall current. The transducer arrays 10 themselves in this embodiment are similar to the transducer arrays 10 described above. But the AC voltages are applied to those transducer arrays 10 in a different pattern, as described immediately below.

In this FIG. 4 embodiment, an AC signal generator 20 generates an AC output signal that has two phases, each of which is input to a bank of switches 25. A variety of approaches for implementing the bank of switches 25 will be apparent to persons skilled in the relevant arts (including but not limited to wiring four SPST solid state relays between the first-phase input and each of the four outputs, respectively, plus an additional four SPST solid state relays between the second-phase input and each of the four outputs, respectively). The path that the AC output signal takes when traveling through the bank of switches 25 is controlled by signals that originate in a controller 30. The bank of switches 25 (operating in response to commands from the controller 30) is configured to route one phase of the AC output signal to two of the transducer arrays 10 simultaneously, and to route the other phase of the AC output signal to the other two transducer arrays 10 simultaneously.

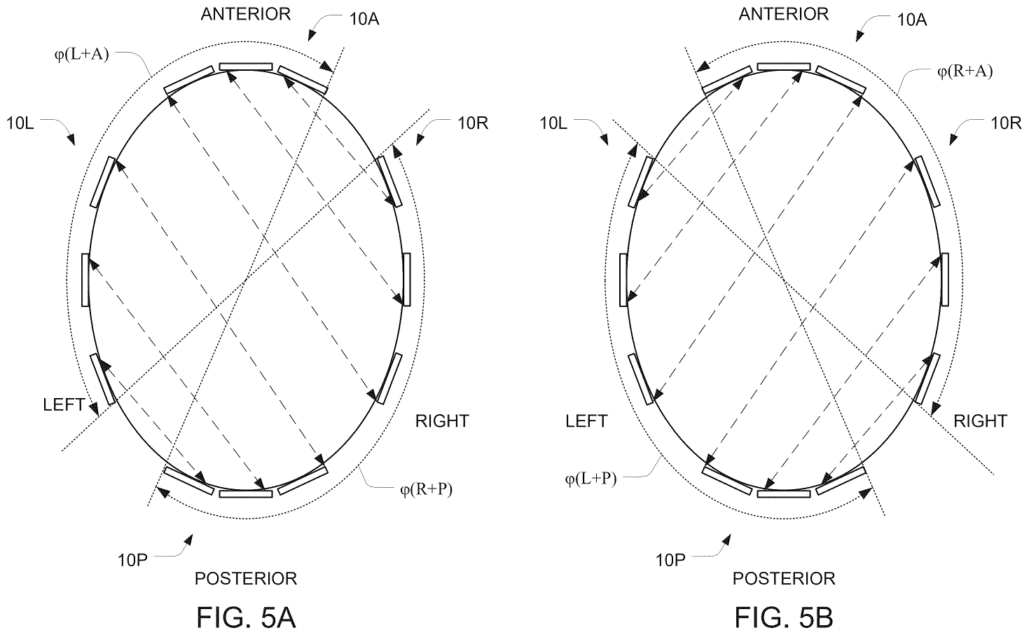

For example, the bank of switches 25 could be commanded to route one phase of the AC output signal to both the anterior and left transducer arrays 10A, 10L, and to route the other phase of the AC signal to both the posterior and right arrays 10P, 10R. When the bank of switches 25 is configured in this manner, these voltages will induce an electric field in the subject's head with field lines that run, on average, diagonally (i.e., the direction of the dashed lines in FIG. 5A). The bank of switches 25 could also be commanded to route one phase of the AC output signal to both the posterior and left transducer arrays 10P, 10L, and to route the other phase of the AC signal to both the anterior and right arrays 10A, 10R. When the bank of switches 25 is configured in this manner, these voltages will induce an electric field in the subject's head with field lines that run, on average, diagonally (i.e., the direction of the dashed lines in FIG. 5B).

Let us now analyze what happens when the FIG. 4 embodiment is used to energize the transducer arrays on subject #1's head using the same output current as the original example (i.e., 2 A), but when each phase of the AC output signal is routed to two of the transducer arrays 10 simultaneously. As explained above, the total area of all the electrode elements within any given transducer array is 28 cm². But because each phase of the AC output signal is routed to two transducer arrays 10 simultaneously, the same 2 A of current is now spread out over 2×28 cm²=56 cm². The current density at each of the transducer arrays 10 will therefore be 2 A÷56 cm²=36 mA/cm², which is below the 60 mA/cm² electrosensation threshold for subject #1. Indeed, the current in this embodiment could be increased beyond 3 A before reaching subject #1's electrosensation threshold.

As explained above, when each phase of the AC output signal is routed to two transducer arrays 10 simultaneously, the current of the AC output signal is spread out over a significantly larger area. During those times, the spread of the electrode elements that are actively being used at any given instant is significantly wider than the spread of the electrode elements that are active at any given instant in the prior art Optune® system, whether measured in area (as described above) or other units e.g., azimuthal angle or circumference (as described below).

FIGS. 5A and 5B are plan views of a subject's head that is being treated with alternating electric fields using four sets of transducer arrays 10A, 10P, 10L, and 10R, which are positioned on the anterior, posterior, left, and right sides of the subject's head, respectively. The dashed lines in FIG. 5A depict a very rough approximation of the electric field lines when one phase of the AC output signal is routed to both the anterior and left transducer arrays 10A, 10L and the other phase of the AC signal is routed to both the posterior and right arrays 10P, 10R. In this situation, the anterior and left transducer arrays collectively operate as a first set of electrode elements, and the posterior and right transducer arrays collectively operate as a second set of electrode elements.

In this situation, an alternating electric field is applied to the subject's head by applying an alternating voltage between (a) the first set of one or more electrode elements 10A, 10L positioned on a first side of the head and (b) the second set of one or more electrode elements 10P, 10R positioned on or in a second side of the head. The first side and the second side are on opposite sides of the longitudinal axis of the head.

Notably, the first set of one or more electrode elements collectively subtend a first azimuthal angle φ(L+A) with respect to the longitudinal axis, and the second set of one or more electrode elements collectively subtend a second azimuthal angle φ(R+P) with respect to the longitudinal axis. (Note that the azimuthal angles referred to herein are measured in polar coordinates in a plane that is perpendicular to the longitudinal axis of the head.)

The first and second azimuthal angles φ(L+A) and φ(R+P) in FIG. 5A are each at least 120°, and in some embodiments each of these angles is at least 135°. These angles are significantly larger than the azimuthal angles collectively subtended by the electrode elements that are active at any given instant in the prior art Optune® system (i.e., φ(L) and φ(R) in FIG. 3A at instants in time when an AC voltage is being applied between the left and right transducer arrays 10L and 10R, or φ(A) and φ(P) in FIG. 3B at instants in time when an AC voltage is being applied between the anterior and posterior transducer arrays 10A and 10P).

A similar situation exists when the circumference of the head that is spanned by the electrode elements within each of the first and second sets of electrode elements is considered. More specifically, the first set of one or more electrode elements collectively span more than 15 cm when measured around the circumference of the head, and the second set of one or more electrode elements collectively span more than 15 cm when measured around the circumference of the head. These distances are significantly larger than the circumference that is collectively spanned by the electrode elements that are active at any given instant in the prior art Optune® system for the left and right transducer arrays 10L and 10R at instants in time when an AC voltage is being applied between those arrays, as depicted in FIG. 3A. In some embodiments, these distances are even larger, and each of the first and second sets of one or more electrode elements collectively span more than 18, 20, or 25 cm when measured around the circumference of the head.

Regardless of whether the span of the electrode elements that are active at any given instant is measured in azimuthal angles or in centimeters along a circumference, the electrode elements cover at least one third of the span that runs from the beginning to the end of the arc φ(L+A), and the electrode elements cover at least one third of the span that runs from the beginning to the end of the arc φ(R+P). (This means that the empty spaces between the electrode elements cover less than two thirds of the span.) In some embodiments, the electrode elements cover at least one half of the span that runs from the beginning to the end of the arc φ(L+A), and the electrode elements cover at least one half of the span that runs from the beginning to the end of the arc φ(R+P). (This means that the empty spaces between the electrode elements cover less than half of the span.) The coverage within those spans could even be higher (e.g., >65%, >80%, or even 100%).

The situation depicted in FIG. 5B is similar to the situation described above in connection with FIG. 5A, except that the dashed lines in FIG. 5B depict a very rough approximation of the electric field lines when one phase of the AC output signal is routed to both the anterior and right transducer arrays 10A, 10R and the other phase of the AC signal is routed to both the posterior and left arrays 10P, 10L. In this situation, the anterior and right transducer arrays collectively operate as the first set of electrode elements, and the posterior and left transducer arrays collectively operate as the second set of electrode elements.

In some circumstances, it can be advantageous to repeatedly alternate the direction of the alternating electric field between the direction depicted in FIG. 5A and the direction depicted in FIG. 5B. One example is when the alternating electric fields are TTFields that are being used to treat a tumor. (This is because the sensitivity of tumors to electric fields is directional.) Another example is when repeatedly shifting the direction of the alternating electric field will increase the coverage of the field within the head based on the geometry of the transducer arrays with respect to the head. The hardware depicted in FIG. 4 can implement this alternation between the FIG. 5A direction and the FIG. 5B direction as described immediately below.

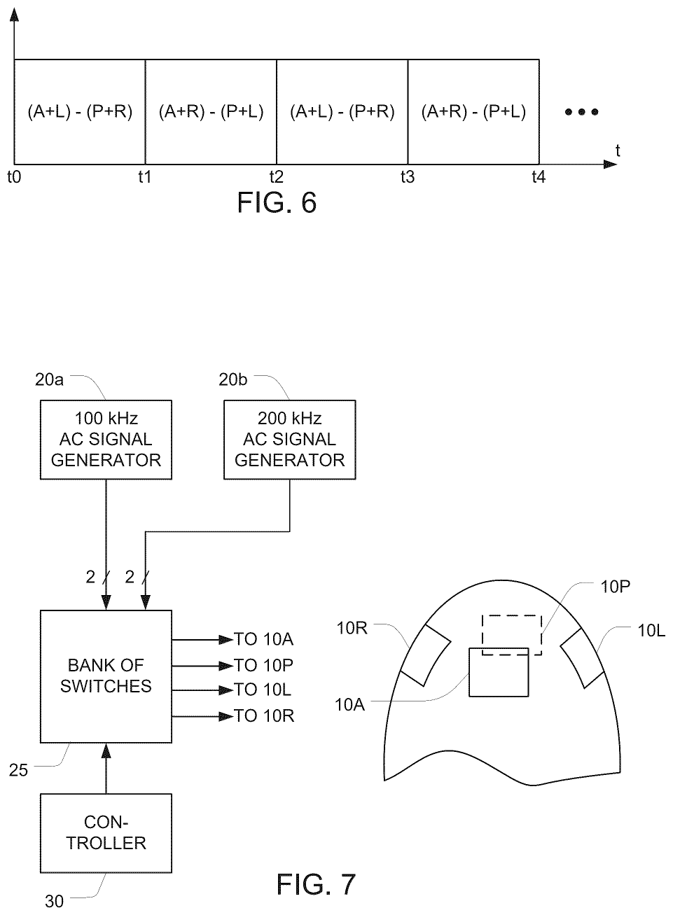

FIG. 6 depicts the commands that are issued by the controller 30 to implement this alternation. First, between t0 and t1, the controller 30 issues commands to the bank of switches 25 to route one phase of the AC output signal to both the anterior and left transducer arrays 10A, 10L, and to route the other phase of the AC signal to both the posterior and right arrays 10P, 10R, which induces an electric field in the FIG. 5A direction. Then, between t1 and t2, the controller 30 issues commands to the bank of switches 25 to route one phase of the AC output signal to both the posterior and left transducer arrays 10P, 10L, and to route the other phase of the AC signal to both the anterior and right arrays 10AP, 10R, which induces an electric field in the FIG. 5B direction. These two steps are then repeated in an alternating sequence at least 10 times. The duration of each step in the sequence could be anywhere between 10 ms and 12 hours. In some embodiments, the duration of each step in the sequence is between 100 ms and 1000 s, or between 200 ms and 5 s (e.g., 1 s).

The hardware depicted in FIG. 4 can therefore be used to practice a method for applying an alternating electric field to a target region in a subject's body using a first set of one or more electrode elements 10A positioned at a first side of the target region, a second set of one or more electrode elements 10P positioned at a second side of the target region, a third set of one or more electrode elements 10L positioned at a third side of the target region, and a fourth set of one or more electrode elements 10R positioned at a fourth side of the target region. This method comprises (a) applying an alternating voltage at a first frequency between (i) both the first and third sets 10A, 10L of one or more electrode elements and (ii) both the second and fourth 10P, 10R sets of one or more electrode element; and (b) applying an alternating voltage at a second frequency between (i) both the first and fourth sets 10A, 10R of one or more electrode elements and (ii) both the second and third sets 10P, 10L of one or more electrode elements. In this method, (1) the first side and the second side are on opposite sides of the target region; (2) the third side and the fourth side are on opposite sides of the target region; (3) the third side is disposed circumferentially between the first side and the second side; (4) the fourth side is disposed circumferentially between the first side and the second side; and (5) steps (a) and (b) are repeated in an alternating sequence at least ten times.

At some point in time prior to practicing this method, the first, second, third, and fourth sets of one or more electrode elements 10 are all positioned on or in the subject's body. Optionally, the first and second frequencies may each be between 50 kHz and 1 MHZ, for example between 100 kHz and 500 kHz or between 100 kHz and 300 kHz. In some embodiments the first and second frequencies may be the same.

This method can be used to apply an alternating electric field to a variety of different body parts, including but not limited to the thorax, abdomen and the head. When the body part is a head, the collective area of the first set of one or more electrode elements may be at least 40 cm², and the collective area of the second set of one or more electrode elements may be at least 40 cm².

As noted above, alternating electric fields at certain frequencies (e.g., 100-500 kHz) can be used to treat tumors, and alternating electric fields at typically lower frequencies (e.g., at 50-200 kHz) can increase the permeability of the blood brain barrier (BBB) e.g., to help chemotherapy drugs reach the brain. Combining alternating electric fields at the former frequencies with chemotherapy that is augmented by alternating electric fields at the latter frequencies can therefore be an effective approach to treating a brain tumor. See, e.g., U.S. Pat. No. 10,967,167, which is incorporated herein by reference in its entirety.

The presence and intensity of electrosensation is an intensity-dependent effect with unpleasant electrosensations typically being worse at higher field intensities. The presence and intensity of electrosensation is also a frequency-dependent effect. More specifically, all other things being equal, the unpleasant electrosensations associated with alternating electric fields will typically be worse at the lower frequencies that are effective at increasing the permeability of the BBB (e.g., 100 kHz) as compared to the frequencies that are most effective at treating tumors (e.g., 200 kHz for glioblastoma).

Let us assume that we encounter a subject with a glioblastoma, and we want to treat that subject with 200 kHz TTFields. If we position four transducer arrays on the subject's head as depicted in FIGS. 2A-D, we can treat the glioblastoma using the conventional field-direction switching approach by (a) applying a 200 kHz AC voltage between arrays 10L, 10R for 1 second, which induces an electric field through the tumor with field lines that run, on average, from side to side through the subject's head as shown in FIG. 3A; then (b) applying a 200 kHz AC voltage between the arrays 10A, 10P for 1 second, which induces an electric field through the tumor with field lines that run, on average, from front to back as shown in FIG. 3B; then repeating that two-step sequence (a) and (b) for e.g., 12-16 hours each day.

Let us also assume that we want to use an alternating electric field at 100 kHz to increase the permeability of the same subject's BBB, to increase the efficacy of chemotherapy drugs that are administered e.g., every few weeks. If we continue using the conventional field-direction switching approach described in the previous paragraph and simply lower the frequency of the alternating electric field to 100 kHz, the subject may experience electrosensation. This is because sensitivity to electrosensation is significantly higher at 100 kHz than at 200 kHz.

FIG. 7 is a block diagram of an embodiment that uses the conventional approach to apply 200 kHz alternating electric fields in the L/R and A/P directions, and also uses the transducer-array-pairing concepts described above in connection with FIG. 4-6 to reduce the current density at each of the transducer arrays while outputting the 100 kHz alternating electric fields. The transducer arrays 10 themselves in this embodiment are similar to the transducer arrays 10 described above. But the AC voltages are applied to those transducer arrays 10 in a different pattern, as described immediately below.

In this FIG. 7 embodiment, the first AC signal generator 20a generates a 100 kHz AC output signal that has two phases, each of which is input to a bank of switches 25. A variety of approaches for implementing the bank of switches 25 will be apparent to persons skilled in the relevant arts (including but not limited to wiring four SPST solid state relays between the first-phase 100 kHz input and each of the four outputs, respectively, plus an additional four SPST solid state relays between the 100 kHz second-phase input and each of the four outputs, respectively, plus an additional four SPST solid state relays between the first-phase 200 kHz input and each of the four outputs, respectively, plus an additional four SPST solid state relays between the 200 kHz second-phase input and each of the four outputs, respectively). The path that the 100 kHz AC output signal takes when traveling through the bank of switches 25 is controlled by signals that originate in a controller 30. The bank of switches 25 (operating in response to commands from the controller 30) is configured to route one phase of the 100 kHz AC output signal to two of the transducer arrays 10 simultaneously, and to route the other phase of the 100 kHz AC output signal to the other two transducer arrays 10 simultaneously.

For example, the bank of switches 25 could be commanded to route one phase of the 100 kHz AC output signal to both the anterior and left transducer arrays 10A, 10L, and to route the other phase of the 100 kHz AC signal to both the posterior and right arrays 10P, 10R. When the bank of switches 25 is configured in this manner, these voltages will induce an electric field in the subject's head with field lines that run, on average, diagonally (i.e., the direction of the dashed lines in FIG. 5A). The bank of switches 25 could also be commanded to route one phase of the 100 kHz AC output signal to both the posterior and left transducer arrays 10P, 10L, and to route the other phase of the 100 kHz AC signal to both the anterior and right arrays 10A, 10R. When the bank of switches 25 is configured in this manner, these voltages will induce an electric field in the subject's head with field lines that run, on average, diagonally (i.e., the direction of the dashed lines in FIG. 5B).

The FIG. 7 embodiment can therefore be used to ameliorate or eliminate electrosensation at the lower 100 kHz frequency using the approaches described above in connection with FIGS. 4-6. More specifically, the electrosensation can be ameliorated or eliminated by configuring the bank of switches 25 (operating in response to commands from the controller 30) to route one phase of the 100 kHz AC output signal to two of the transducer arrays 10 simultaneously, and to route the other phase of the 100 kHz AC output signal to the other two transducer arrays 10 simultaneously. Operating the transducer arrays in pairs in this matter will reduce the current density at each of the transducer arrays, which will eliminate or ameliorate electrosensation at 100 kHz.

In addition to routing the 100 kHz AC signal from signal generator 20a to the four selected outputs as described above, the bank of switches 25 is also configured to route the 200 kHz AC signal from signal generator 20b to the four selected outputs via different paths to (a) apply the 200 kHz AC voltage between arrays 10L, 10R for e.g., 1 second, which induces an electric field through the tumor with field lines that run, on average, from side to side through the subject's head as shown in FIG. 3A; then (b) apply a 200 kHz AC voltage between the arrays 10A, 10P for e.g., 1 second, which induces an electric field through the tumor with field lines that run, on average, from front to back as shown in FIG. 3B; then repeating that two-step sequence (a) and (b) for e.g., 12-16 hours each day. To do this, the controller 30 commands the bank of switches 25 to route one phase of the 200 kHz AC output signal to the left transducer array 10L and to route the other phase of the 200 kHz AC signal to the right array 10R during certain times. When the bank of switches 25 is configured in this manner, these voltages will induce an electric field in the subject's head with field lines that run, on average, left to right (i.e., the direction of the dashed lines in FIG. 3A). During other times, the controller 30 commands the bank of switches 25 to route one phase of the 200 kHz AC output signal to anterior transducer arrays 10A and to route the other phase of the 200 kHz AC signal to the posterior array 10P. When the bank of switches 25 is configured in this manner, these voltages will induce an electric field in the subject's head with field lines that run, on average, front to back (i.e., the direction of the dashed lines in FIG. 3B).

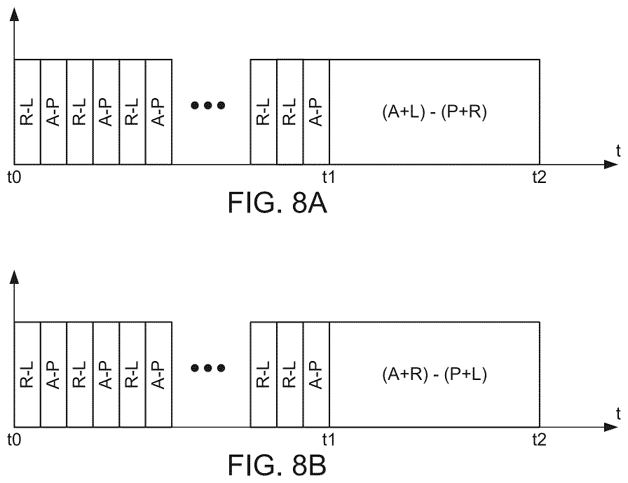

FIG. 8A depicts one suitable set of commands that may be issued by the controller 30 in FIG. 7 to implement this approach. During the window of time between to and t1, the controller 30 (a) issues commands to the bank of switches 25 to route one phase of the first (e.g., 200 kHz) AC output signal the left transducer arrays 10L, and to route the other phase of the first (e.g., 200 kHz) AC signal to the right transducer arrays 10R, which induces an electric field in the FIG. 3A direction for a duration of time (e.g., 1 second); then (b) issues commands to the bank of switches 25 to route one phase of the second (e.g., 200 kHz) AC output signal the anterior transducer arrays 10A, and to route the other phase of the second (e.g., 200 kHz) AC signal to the posterior transducer arrays 10P, which induces an electric field in the FIG. 3B direction for a duration of time (e.g., 1 second). This two-step sequence (a) and (b) is then repeated for e.g., 12-16 hours each day until t1. Then, during the window of time between t1 and t2 (a window that is preferably synchronized in time with the administration of chemotherapy), the controller 30 issues commands to the bank of switches 25 to route one phase of the third (e.g., 100 kHz) AC output signal to both the anterior and left transducer arrays 10A, 10L, and to route the other phase of the third (e.g., 100 kHz) AC signal to both the posterior and right arrays 10P, 10R, which induces an electric field in the FIG. 5A direction.

FIG. 8B depicts another suitable set of commands that may be issued by the controller 30 (in FIG. 4) to implement this approach. The operation of the controller during the window of time between to and t1 is the same as described above in connection with FIG. 8A. Then, during the window of time between t1 and t2 (a window that is preferably synchronized in time with the administration of chemotherapy), the controller 30 issues commands to the bank of switches 25 to route one phase of the third (e.g., 100 kHz) AC output signal to both the anterior and right transducer arrays 10A, 10R, and to route the other phase of the third (e.g., 100 kHz) AC signal to both the posterior and left arrays 10P, 10L, which induces an electric field in the FIG. 5B direction.

Returning to FIG. 7, as an alternative to using separate 100 kHz and 200 kHz AC signal generators 20a, 20b as depicted in FIG. 7, a single multi-frequency AC signal generator (not shown) that is capable of operating at either 100 kHz or 200 kHz can be used. In these embodiments, the single multi-frequency AC signal generator should be configured to respond to a frequency-setting command from the controller, so that the correct frequency will arrive at the correct output at the appropriate times, as described above in connection with FIGS. 8A and 8B.

When implementing the approaches described above in connection with either FIG. 8A or 8B, the hardware depicted in FIG. 4 can be used to practice a method for treating a tumor in a target region in the subject's head and delivering a substance across the BBB in the target region. This method comprises applying an alternating voltage at a first frequency (e.g., 200 kHz) between a first set of one or more electrode elements 10A positioned at a first side of the target region and a second set of one or more electrode elements 10P positioned at a second side of the target region, wherein the first side and the second side are on opposite sides of the target region. This method also comprises applying an alternating voltage at a second frequency (e.g., 200 kHz) between a third set of one or more electrode elements 10L positioned at a third side of the target region and a fourth set of one or more electrode elements 10R positioned at a fourth side of the target region, wherein the third side and the fourth sides are on opposite sides of the target region, wherein the third side is disposed circumferentially between the first side and the second side, and wherein the fourth side is disposed circumferentially between the first side and the second side. And this method also comprises applying an alternating voltage at a third frequency (e.g., 100 kHz) between (i) both the first and third sets of one or more electrode elements 10A, 10L and (ii) both the second and fourth sets of one or more electrode elements 10P, 10R, wherein the third frequency is lower than the first frequency and the third frequency is lower than the second frequency. The alternating voltage at the first frequency, the alternating voltage at the second frequency, and the alternating voltage at the third frequency are each applied during different intervals of time.

At some point in time prior to practicing this method, the first, second, third, and fourth sets of one or more electrode elements 10 are all positioned at the first, second, third, and fourth sides of the target region, respectively. This may be accomplished, e.g., by positioning the sets of electrode elements on the subject's skin, or implanting them beneath the subject's skin.

In some embodiments of this method, the first frequency (e.g., 200 kHz) is selected so that an electric field that is induced in the target region due to application of the alternating voltage between the first set of one or more electrode elements 10A and the second set of one or more electrode elements 10P will have an anti-mitotic effect. The second frequency (e.g., 200 kHz) is selected so that an electric field that is induced in the target region due to application of the alternating voltage between the third set of one or more electrode elements 10L and the fourth set of one or more electrode elements 10R will have an anti-mitotic effect. And the third frequency (e.g., 100 kHz) is selected so that an electric field that is induced in the target region due to application of the alternating voltage at the third frequency between (a) both the first and third sets of one or more electrode elements 10A, 10L and (b) both the second and fourth sets of one or more electrode elements 10P, 10R will increase permeability of the BBB in the target region.

Optionally, the first frequency and the second frequency are the same. For example, when treating glioblastoma, the first and second frequencies could both be 200 kHz. In some embodiments, the first and second frequencies are each between 50 kHz and 1 MHz, and the third frequency is between 50 kHz and 300 kHz. In some embodiments, the alternating voltage at the third frequency is applied for at least 24 hours.

In some embodiments, the steps of (a) applying the alternating voltage at the first frequency between the first and second sets of one or more electrode elements 10A, 10P and (b) applying the alternating voltage at the second frequency between the third and fourth sets of one or more electrode elements 10L, 10R are repeated in an alternating sequence at least 10,000 times. Assuming that step (a) and step (b) take 1 second each, this corresponds to a total of 20,000 seconds (i.e., about 5.5 hours)

While the discussion above focusses on the context of applying alternating electric fields to a subject's head, similar approaches can be used in other parts of the body, including but not limited to organs within the torso.

Portions of the discussion above focus on applying alternating electric fields to a subject's head in order to increase the permeability of the blood brain barrier. Similar concepts can be applied to other biological barriers that are physically located in other parts of the body (e.g., the intestinal epithelial barrier).

When alternating electric fields are used to treat a tumor, it is preferable to use an electric field whose orientation alternates between different directions, as described above. But when alternating electric fields are used for other purposes (e.g., increasing the permeability of the blood brain barrier or another biological barrier), an electric field whose orientation remains constant can be used.

In some embodiments, each of the electrode elements within each of the transducer arrays 10L, 10R, 10A, and 10P is a capacitively-coupled electrode element (i.e., a conductive plate covered by a layer of a dielectric material on the side that faces the subject's body). But in alternative embodiments, they can be conductive electrode elements (e.g., a flat slab of metal).

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method for treating a tumor in a target region and delivering a substance across a biological barrier in the target region, the method comprising:
applying an alternating voltage at a first frequency between a first set of one or more electrode elements positioned at a first side of the target region and a second set of one or more electrode elements positioned at a second side of the target region, wherein the first side and the second side are on opposite sides of the target region;
applying an alternating voltage at a second frequency between a third set of one or more electrode elements positioned at a third side of the target region and a fourth set of one or more electrode elements positioned at a fourth side of the target region, wherein the third side and the fourth sides are on opposite sides of the target region, wherein the third side is disposed circumferentially between the first side and the second side, and wherein the fourth side is disposed circumferentially between the first side and the second side; and
applying an alternating voltage at a third frequency between (i) both the first and third sets of one or more electrode elements and (ii) both the second and fourth sets of one or more electrode elements, wherein the third frequency is lower than the first frequency and the third frequency is lower than the second frequency,
wherein the alternating voltage at the first frequency, the alternating voltage at the second frequency, and the alternating voltage at the third frequency are each applied during different intervals of time.

2. The method of claim 1, further comprising:
positioning the first set of one or more electrode elements at the first side of the target region;
positioning the second set of one or more electrode elements at the second side of the target region;
positioning the third set of one or more electrode elements at the third side of the target region; and
positioning the fourth set of one or more electrode elements at the fourth side of the target region.

3. The method of claim 1, wherein the first frequency is selected so that an electric field that is induced in the target region due to application of the alternating voltage at the first frequency between the first set of one or more electrode elements and the second set of one or more electrode elements will have an anti-mitotic effect,
wherein the second frequency is selected so that an electric field that is induced in the target region due to application of the alternating voltage at the second frequency between the third set of one or more electrode elements and the fourth set of one or more electrode elements will have an anti-mitotic effect, and
wherein the third frequency is selected so that an electric field that is induced in the target region due to application of the alternating voltage at the third frequency between (a) both the first and third sets of one or more electrode elements and (b) both the second and fourth sets of one or more electrode elements will increase permeability of the biological barrier in the target region.

4. The method of claim 3, wherein the first frequency and the second frequency are the same.

5. The method of claim 1, wherein the first and second frequencies are each between 50 kHz and 1 MHz, and wherein the third frequency is between 50 kHz and 300 kHz.

6. The method of claim 1, wherein the alternating voltage at the third frequency is applied for at least 24 hours.

7. The method of claim 1, wherein the steps of (a) applying the alternating voltage at the first frequency between the first set of one or more electrode elements and the second set of one or more electrode elements and (b) applying the alternating voltage at the second frequency between the third set of one or more electrode elements and the fourth set of one or more electrode elements are repeated in an alternating sequence at least 10,000 times.

8. A method for applying an alternating electric field to a target region in a subject's body using a first set of one or more electrode elements positioned at a first side of the target region, a second set of one or more electrode elements positioned at a second side of the target region, a third set of one or more electrode elements positioned at a third side of the target region, and a fourth set of one or more electrode elements positioned at a fourth side of the target region, the method comprising:
(a) applying an alternating voltage at a first frequency between (i) both the first and third sets of one or more electrode elements and (ii) both the second and fourth sets of one or more electrode elements; and
(b) applying an alternating voltage at a second frequency between (i) both the first and fourth sets of one or more electrode elements and (ii) both the second and third sets of one or more electrode elements,
wherein the first side and the second side are on opposite sides of the target region,
wherein the third side and the fourth side are on opposite sides of the target region,
wherein the third side is disposed circumferentially between the first side and the second side,
wherein the fourth side is disposed circumferentially between the first side and the second side, and
wherein steps (a) and (b) are repeated in an alternating sequence at least ten times.

9. The method of claim 8, further comprising:
positioning the first set of one or more electrode elements on or in the subject's body;
positioning the second set of one or more electrode elements on or in the subject's body;
positioning the third set of one or more electrode elements on or in the subject's body; and
positioning the fourth set of one or more electrode elements on or in the subject's body.

10. The method of claim 8, wherein the first frequency and the second frequency are each between 50 kHz and 1 MHz.

11. The method of claim 8, wherein the first frequency and the second frequency are the same.

12. An apparatus for applying signals to a set of electrodes, the apparatus comprising:
at least one signal generator configured to generate an alternating voltage;
a bank of switches having at least one control input, a first output terminal, a second output terminal, a third output terminal, and a fourth output terminal,
wherein the bank of switches inputs the alternating voltage at the first frequency,
wherein the bank of switches is configured to, in response to a first state of the at least one control input, route the alternating voltage so that the alternating voltage appears between (a) both the first output terminal and the third output terminal and (b) both the second output terminal and the fourth output terminal, and
wherein the bank of switches is configured to, in response to a second state of the at least one control input, route the alternating voltage so that the alternating voltage appears between (a) both the first output terminal and the fourth output terminal and (b) both the second output terminal and the third output terminal; and
a controller programmed to repeat the following steps in an alternating sequence at least ten times (i) set the at least one control input to the first state, and (ii) set the at least one control input to the second state.

13. The apparatus of claim 12, wherein the alternating voltage has a frequency between 50 kHz and 1 MHz.
